(19) United States
(12) Patent Application Publication
(10) Pub. No.: US 2026/0335049 A1
(43) Pub. Date: Oct. 29, 2026

(54) TRANSGINGIVAL DENTAL IMPLANT AND PROSTHETIC SYSTEM USING SUCH AN IMPLANT

(71) Applicant: AXENTISS, Toulouse (FR)

(72) Inventors: Luc TREVISAN, Toulouse (FR); Régis NEGRE, Onet-le-Chateau (FR)

(21) Appl. No.: 19/487,267

(22) PCT Filed: May 21, 2024
(86) PCT No.: PCT/EP2024/063975

§ 371 Date: Nov. 24, 2025

(30) Foreign Application Priority Data
May 26, 2023 (FR) 2305281

(51) Int. Cl. A61C 8/00; A61C 13/00

(52) U.S. Cl. CPC A61C 8/0015; A61C 8/0022; A61C 13/0006

(57) ABSTRACT

A transgingival dental implant extending longitudinally along a first smooth section intended to lie flush in the oral cavity of a patient at a first end, referred to as the cervical end, and along a second section provided with a male thread, continuing the first section, the threaded section being intended to be screwed into the bone of the jaw of the patient and defining a second end, referred to as the apical end, the implant defining a body of revolution along the longitudinal axis and further including an inner connection housing that opens out at the first, cervical end and is intended to receive the means for fastening a screw-retained dental component, the implant is coated, on at least the smooth section, with a thin layer of zirconia.

The present invention relates to a transgingival dental implant, as well as a prosthetic system using such an implant, said prosthetic system comprising the implant, an abutment-type dental component and a dental prosthesis.

Oral implantology is a surgical technique designed to treat a dental pathology—edentulism—by replacing one or more teeth in edentulous patients with a dental implant. This technique involves implanting an artificial root made of biocompatible materials into the maxillary or mandibular bone.

The etiological causes of this pathology can be diverse. The consequences of edentulism depend on the extent to which one or more teeth are missing, and can vary. This can result in altered normal physiology, increased risk of impaired mastication, deterioration of oral health, a higher prevalence of obesity, increased risk of cardiovascular disease and gastrointestinal disorders, increased risk of chronic gastric inflammation, pancreatic and gastrointestinal cancers and ulcers, increased risk of non-insulin-dependent diabetes, reduced health-related quality of life, and an association between edentulism and sleep-disordered breathing (such as obstructive sleep apnea). The population affected by edentulism is very broad, but it particularly affects the elderly.

Implantology can be used to treat single-tooth edentulism, i.e. the loss of one tooth; multiple tooth edentulism, i.e. the loss of several adjacent or non-adjacent teeth; or total edentulism, i.e. the loss of all teeth. The implant is used to support a crown, a partial or total bridge, or a total removable prosthesis.

Implantology is a promising alternative to conventional treatments such as denture bridges or partial or total removable appliances, which can provide a satisfactory response to the dental pathology of edentulism in the short to medium term. In fact, dental implants deliver better results and are more durable over the long term.

Thus, patients treated with implant reconstructions showed improvement in masticatory function, speech function, and overall quality of life.

The fundamental characteristics of an implant system, together with the various surgical procedures used and the characteristics of the materials used in oral implantology, will now be outlined.

An implant system is therefore a trio of components, comprising the implant itself, the prosthetic fittings, and the ancillary surgical instruments needed to create an implant restoration.

There are two surgical approaches.

The two-stage approach means that the implant is embedded in the bone and covered by the gingiva while the bone heals. Once the bone has healed, the gingiva above the implant is incised to expose the implant, and a transgingival healing abutment is attached, allowing the gingiva to heal. In this case, the gingiva will heal around the healing abutment and not around the implant. After gingival healing, this abutment is removed and replaced by a prosthetic element to which the crown is attached. This is referred to as gingival healing by secondary intention.

The one-stage approach involves placing an implant with a different design, since it will have both an endosseous and a transgingival part. In this configuration, gingival healing by primary intention is achieved around the transgingival part of the implant. Epithelial attachment is a permanent feature of this technique. This technique, wherein the implant is immediately connected to the oral cavity, does not require a second operation.

In addition, the dental implants are made from perfectly biocompatible biomaterials. The properties of these biomaterials, combined with a specific surface finish, induce a natural colonization of neoformed bone on the implant surface once the implant has been inserted into the jawbone. This process, called osseointegration and highlighted by Professor Per-Ingvar Brånemark, is defined as the direct structural and functional connection between living bone and the surface of an artificial implant.

The vast majority of dental implants are made from alloyed titanium, such as Grade 5 Ti6Al4V, or unalloyed titanium, such as Grade 4 T60. New ranges of implants machined from sintered zirconia blanks have emerged, so that 80% of implants on the market are made of titanium and 20% of zirconia.

Historically, the titanium range has been the material of choice for implant restorations. However, in rare cases, this material can be allergenic and therefore less well tolerated by tissues. Because of its metallic color, it is also unsightly and results in compromised aesthetics due to its grayish appearance showing through the gingival tissues.

Ceramic implants are also available, mainly in alumina (Al2O3) or zirconia (ZrO2). They are not subject to electrochemical corrosion, but despite the latest advances, their impact resistance is ten times lower than that of titanium alloys. As a result, the use of these materials has less scientific backing, particularly in terms of mechanical strength, to compete with titanium implants. Indeed, the Young's modulus (or modulus of elasticity) is twice as high for sintered Y-TZP zirconia (200 GPa) as for grade 4 titanium (100 GPa).

The purpose of the present invention is therefore to offer a transgingival dental implant with specific surface treatments to improve the biological response of each of the tissues involved to the foreign body represented by the implant.

By understanding the three strategic zones of bone, gingiva, and prosthetics, the inventors sought to optimize the prosthetic system in terms of surface treatments.

The subject matter of the invention is in particular a transgingival dental implant extending longitudinally along a first, smooth section intended to lie flush in the oral cavity of a patient at a first end, referred to as the cervical end, and along a second section provided with a male thread, continuing the first section, said threaded section being intended to be screwed into the jawbone of said patient and defining a second end, referred to as the apical end, the implant defining a body of revolution along the longitudinal axis and further comprising an inner connection housing that opens out at the first, cervical end and is intended to receive the means for fastening a screw-retained dental component, the implant being coated, on at least the smooth section, with a thin layer of zirconia.

Optional, complementary, or alternative features of the invention are set out below.

The thin layer of zirconia is preferably produced using a chemical vapor deposition (CVD) process.

Alternatively, the thin layer of zirconia is preferably produced using a thermal spray deposition process.

In another advantageous embodiment, the thin layer of zirconia produced by the chemical vapor deposition or thermal spraying process is a stabilized yttriated zirconia doped with alumina.

Preferably, the thin layer of zirconia produced by the chemical vapor deposition or thermal spraying process is doped with inorganic antimicrobial agents with high antibacterial activity, excellent biocompatibility and sufficient stability, such as silver, copper, zinc, fluorine, calcium and nitrogen.

Even more preferably, the thin layer of zirconia produced by the chemical vapor deposition or thermal spray process is doped with inorganic antimicrobial agents in metallic, ionic or oxide form, such as silver (Ag, Ag⁺, Ag2O), copper (Cu, Cu²⁺, CuO), or zinc (Zn, Zn²⁺, ZnO).

The thin layer of zirconia is preferably between 10 and 100 microns thick.

The thin layer of zirconia preferably has an arithmetic mean roughness, Ra, of less than 0.50 microns, preferably less than 0.20 microns, even more preferably less than 0.10 microns.

Preferably, the smooth section has a portion adjacent to the threaded section, designed to be embedded in the jawbone of said patient.

The adjacent portion is preferably cylindrical or frustoconical in shape, and extends longitudinally over a length of between 1 and 4 mm.

The second threaded section preferably has a surface finish such that the arithmetic mean roughness, Ra, is between 1.2 and 1.8 microns, preferably of the order of 1.5 microns.

The length of the threaded section is preferably about twice the length of the smooth section.

Another object of the invention is a prosthetic system comprising a transgingival dental implant in accordance with one embodiment of the invention, together with an abutment-type screw-retained dental component, and a dental prosthesis.

Other benefits and features of the invention shall become evident upon examining the detailed description of entirely non-limiting implementations and embodiments, and from the following enclosed drawings:

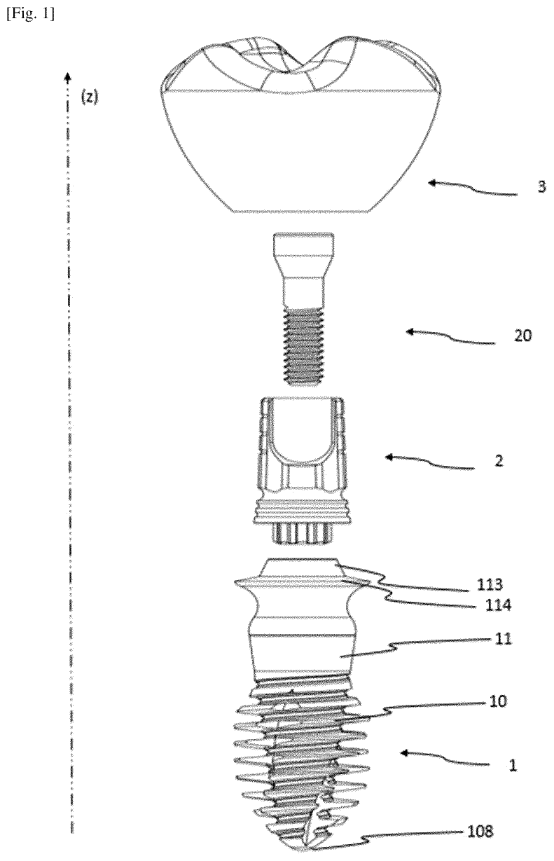

FIG. 1 This figure shows a schematic exploded view of a prosthetic system according to one embodiment of the invention.

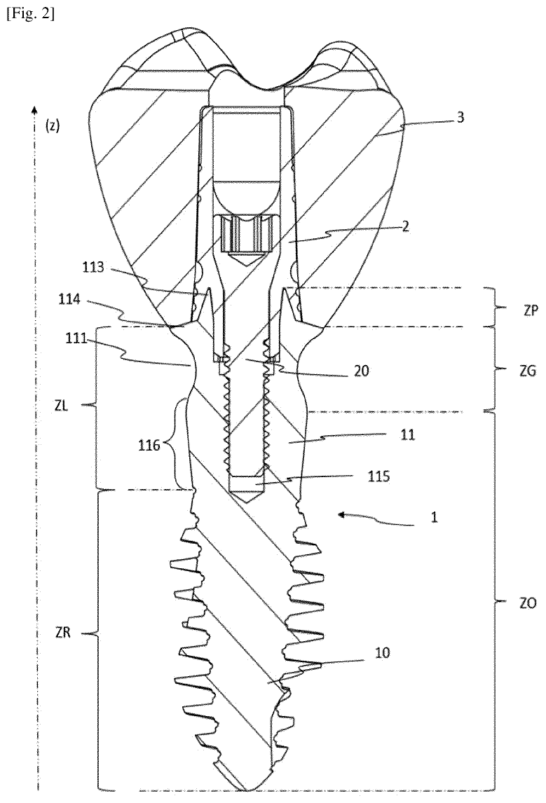

FIG. 2 This figure shows a detailed schematic cross-sectional view of an assembled prosthetic system in accordance with one embodiment of the invention.

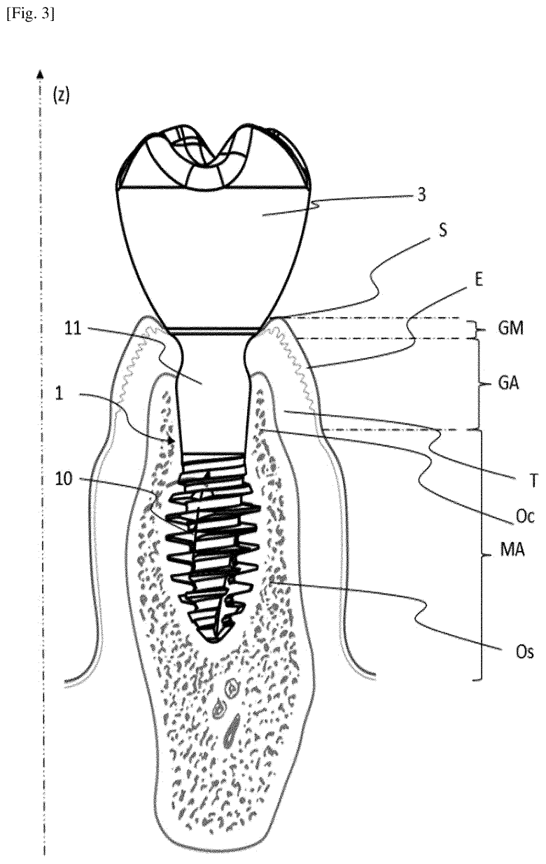

FIG. 3 This figure shows a schematic cross-sectional view of a prosthetic system in accordance with one embodiment of the invention, implanted in the jawbone of a patient.

In relation to FIGS. 1 and 2, the prosthetic system according to the invention comprises a transgingival dental implant 1, together with an abutment-type screw-retained dental component 2 and a dental prosthesis 3.

The transgingival dental implant extends longitudinally along a Z-axis and comprises a smooth first section 11 designed to be flush with a patient's oral cavity at a first, cervical end 114 of the implant.

“Smooth” means that the first section is completely free of asperities. In other words, it has no grooves, ribs, or threads, for example, and low roughness.

The implant further comprises a second section 10 with a male thread, extending the first section, said threaded section being designed to be screwed into the jawbone of said patient and defining the second end, referred to as the apical end 108 of the implant.

The implant defines a body of revolution along the longitudinal axis z and may also comprise a connection appendage 113 extending the first cervical end 114, and an internal connection housing 115 opening out at the first cervical end 114.

The so-called connection appendage 113 strengthens the connection between the implant and a screw-retained dental component 2, such as an abutment, at the part of the prosthetic system that emerges into the patient's oral cavity. Once the implant has been installed in the patient's jaw, the abutment is screwed into the implant and the dental prosthesis 3 is attached (usually by bonding) to the abutment. This creates the prosthetic system.

The housing 115 is designed to accommodate the fastening means 20, such as a screw, of a screw-retained dental component 2, such as an abutment.

According to the principle of the invention, the implant 1 is coated, on at least the smooth section 11, with a thin layer of zirconia, preferably a thin layer of zirconia stabilized with 8% yttrium by weight (yttriated zirconia).

Advantageously, the arithmetic mean roughness, Ra, is less than 0.50 microns, preferably less than 0.20 microns, even more preferably less than 0.10 microns.

Deposition is preferably performed using dry processes.

In an equally preferred embodiment, the thin layer of zirconia is produced using a thermal spray deposition process. “Thermal spray deposition” refers to processes wherein solid materials are broken down into fine particles in a liquid or pasty state in a gas stream to form a stacked coating on a substrate. The carrier gas is used to accelerate and transport fine particles (typically 5 to 100 micrometers), which may be in a liquid, pasty or even solid state, to the substrate. This carrier gas can also be an enthalpy source, enabling these particles to be heated to their melting point. Other processes use an electric arc to melt the material. Depending on their speed, physical state, temperature, or other factors, the particles projected onto the substrate crush together to form splats.

The coating is produced by accumulating and stacking the splats on the substrate.

In another, equally preferred, embodiment, the thin layer of zirconia is produced using a chemical vapor deposition (CVD) process. Chemical vapor deposition is the epitaxial deposition of solid films on the surface of a substrate during the vapor phase of a controlled chemical reaction.

CVD deposition is a fast, versatile method of supporting film growth, enabling the generation of pure coatings of uniform thickness and controlled porosity, even on complex or profiled surfaces. Furthermore, CVD can be applied selectively to specific areas.

Advantageously, the thin layer of zirconia is cation-doped, and preferably doped with metal ions such as silver ions Ag⁺, copper ions Cu²⁺ or zinc ions Zn₂₊. The toxic effect of metal ions, or oligodynamic effect, on microorganism cells, even at relatively low concentrations, drastically reduces the presence and adhesion of microorganisms that are detrimental to implant durability.

Alternatively or additionally, the thin layer of zirconia can be doped with alumina.

Preferably, the thin layer of zirconia has a thickness of between 10 and 100 microns.

The inventors prefer to obtain a sufficiently thick (several tens of microns) and homogeneous layer, particularly on the portion of the smooth section that is flush with the oral cavity, so that the white precursors can be seen by the naked eye, without any transparency effect. This masks the grayish color of the implant, which is usually made of titanium or a titanium alloy.

The inventors also prefer to obtain a sufficiently thick layer over the entire implant.

In this case, the entire implant, in alloyed or unalloyed titanium, is exposed to one or more precursors in the liquid or gas phase, which react on the surface of implant 1 in a reaction chamber to generate the desired doped zirconia deposit.

The resulting zirconia deposit is solid, non-porous, high-performance and highly pure. This zirconia deposit also incorporates the encapsulation of metal cations, such as silver or copper ions. The toxic effect of metal ions, or oligodynamic effect, on microorganism cells, even at relatively low concentrations, drastically reduces the presence and adhesion of microorganisms that are detrimental to implant durability. The zirconia layer thus reduces the risk of periodontal disease and prevents the onset of peri-implantitis.

The hard outer shell of zirconia chemically bonded to the implant surface forms a protective top layer against corrosion. This layer prevents air and moisture from coming into contact with the underlying titanium implant. With oxygen (the cathode) and moisture (the electrolyte) out of the equation, the oxidation reaction cannot take place, and therefore titanium corrosion cannot occur.

The zirconia protection has a high degree of hardness and a very low coefficient of friction. In other words, the zirconia layer enables the implant to be inserted into the tissue, thus reducing frictional forces and the risk of bone heating, which can lead to bone necrosis.

Zirconia has a very low thermal conductivity of around 2.5 W m⁻¹K⁻¹⁸. Its addition to the substrate forms an effective thermal barrier that limits thermal exposure of surrounding tissues. This insulating barrier reduces tissue heating during implant placement.

Thanks to its natural color and smooth surface, the zirconia layer gives the implant a brilliant appearance and durability. This aspect is all the more important in the attachment zone ZG of the gingival tissue. The natural pearly-white appearance obtained is fully comparable to the color of a tooth and its root, proving to be an undeniable aesthetic asset for the patient.

Advantageously, the smooth section 11 has a portion 116 adjacent to the threaded section 10, designed to be embedded in the jawbone of the patient.

Preferably, the adjacent portion 116 is cylindrical or frustoconical in shape (with a taper towards the apical end), and extends over a length of between 1 and 4 mm.

This portion 116 favors the development of distance osteogenesis and corticalization. Bone neoformation, secondary (biological) stability, takes place from the adjacent bone surface, resulting in the formation of woven bone. The newly formed woven bone then matures and remodels itself. The structure first becomes lamellar, then Haversian with permanent remodeling. As the bone remodels and matures, it increasingly becomes mechanically strong.

Preferably, the second threaded section 10 has a surface finish such that the arithmetic mean roughness, Ra, is between 1.2 and 1.8 microns, preferably of the order of 1.5 microns.

Such a rough surface finish can be achieved, particularly when the second threaded section 10 is coated with a thin layer of zirconia, by subtractive techniques such as sandblasting and acid etching, which can be combined.

The advantage of this surface condition is that it encourages contact osteogenesis and bone trabeculation to promote healing in the screw-retained zone.

In summary, and in relation to FIGS. 2 and 3, the implant according to the invention and preferred features, is provided with a smooth section 11 coated with a thin layer of zirconia having very low roughness over a contact depth ZL in the direction of the apex, first with the gingival tissue ZG and then with the bone zone ZO and in particular with the crestal bone Oc.

The implant is also anchored by means of a threaded section 10 with a preferentially higher roughness over a contact depth ZR towards the apex, with the bone zone ZO and in particular with the cancellous bone Os.

The anchoring of the smooth section 11 in the crestal bone Oc by means of the adjacent section 116 helps to protect the gingival attachment zone GA, made up of the peri-implant connective tissue T covered by the juxta-implant epithelium E. The gingival attachment zone GA, located between the marginal gingiva GM and the alveolar mucosa MA, is a particularly sensitive zone.

Preferably, the length ZR of the threaded section 10 is of the order of twice the length ZL of the smooth section 11, with the implant buried to the full depth corresponding to the gingival zone ZG and the bone zone ZO.

In terms of dimensions, the implants can have the following dimensions, with a ZR/ZL ratio of around 2:

| | |
|---|---|
| Length ZR of threaded section | Length ZL of smooth section |

| | | | |
|---|---|---|---|
| 6 | mm | 3 | mm |
| 7.5 | mm | 3.5 | mm |
| 8.5 | mm | 4.5 | mm |
| 10 | mm | 5 | mm |
| 11.5 | mm | 5.5 | mm |

The embodiments described below are in no way limiting, and in particular, it is possible to consider variants of the invention that comprise only a selection of the features disclosed, in isolation from the other features disclosed (even if that selection is isolated within a phrase comprising other features), if this selection of features is sufficient to confer a technical benefit or to differentiate the invention with respect to the prior state of the art. This selection comprises at least one preferably functional feature which lacks structural detail, or only has a portion of the structural details if that portion only is sufficient to confer a technical benefit or to differentiate the invention with respect to the prior state of the art.

Note that the various features, forms, variants and embodiments of the invention may be combined with each other in various combinations as long as they are not incompatible or exclusive of each other.

1. A transgingival dental implant, comprising: said implant extending longitudinally along a first, smooth section designed to lie flush in the oral cavity of a patient at a first end, referred to as the cervical end, and along a second section provided with a male thread, continuing the first section; said threaded section being intended to be screwed into the jawbone of said patient and defining a second end, referred to as the apical end; the implant defining a body of revolution along the longitudinal axis and further comprising an inner connection housing that opens out at the first, cervical end and is intended to receive the means for fastening a screw-retained dental component; and wherein the implant is coated, on at least the smooth section, with a thin layer of zirconia.

2. The transgingival dental implant according to claim 1, characterized in that the thin layer of zirconia is produced using a chemical vapor deposition (CVD) process.

3. The transgingival dental implant according to claim 1, characterized in that the thin layer of zirconia is produced by a thermal spray deposition process.

4. The transgingival dental implant according to claim 2, characterized in that the thin layer of zirconia produced by chemical vapor deposition or thermal spraying is doped, preferably with cations.

5. The transgingival dental implant according to claim 4, characterized in that the thin layer of zirconia produced by a chemical vapor deposition or thermal spraying process is doped with inorganic antimicrobial agents with high antibacterial activity, excellent biocompatibility and sufficient stability, such as silver, copper, zinc, fluorine, calcium, nitrogen.

6. The transgingival dental implant according to claim 5, characterized in that the thin layer of zirconia produced by a chemical vapor deposition process or by thermal spraying is doped with inorganic antimicrobial agents in metallic, ionic or oxide form, such as silver (Ag, Ag⁺, Ag2O), copper (Cu, Cu²⁺, CuO), or zinc (Zn, Zn²⁺, ZnO).

7. The transgingival dental implant according to claim 1, characterized in that the thin layer of zirconia produced by a chemical vapor deposition process or by thermal spraying is a stabilized yttriated zirconia doped with alumina.

8. The transgingival dental implant according to claim 1, characterized in that the thin layer of zirconia has a thickness of between 10 and 100 microns.

9. The transgingival dental implant according to claim 1, characterized in that the thin layer of zirconia has an arithmetic mean roughness, Ra, of less than 0.50 microns.

10. The transgingival dental implant according to claim 1, characterized in that the smooth section has a portion adjacent to the threaded section, intended to be embedded in the jawbone of said patient.

11. The transgingival dental implant according to claim 1, characterized in that the adjacent portion is cylindrical or frustoconical in shape, and extends longitudinally over a length of between 1 and 4 mm.

12. The transgingival dental implant according to claim 1, characterized in that the second threaded section has a surface finish such that the arithmetic mean roughness, noted Ra, is between 1.2 and 1.8 microns.

13. The transgingival dental implant according to claim 1, characterized in that the length of the threaded section is of the order of twice the length of the smooth section.

14. A prosthetic system comprising a transgingival dental implant according to claim 1, together with an abutment-type screw-retained dental component and a dental prosthesis.
